(19) United States
(12) Patent Application Publication
(10) Pub. No.: US 2007/0067033 A1
(43) Pub. Date: Mar. 22, 2007

(54) Hair replacement method

(76) Inventor: Alfred O. Bonati, St. Petersburg, FL (US)

Correspondence Address: J. WILEY HORTON, ESQUIRE;Pennington, Moore, Wilkinson, Bell & Dunbar, P.A., Post Office Box 10095, Tallahassee, FL, 32302-2095, US

(21) Appl. No.: 11/522,662

(22) Filed: Sep. 18, 2006

Related U.S. Application Data
(60) Provisional application No. 60/718,101, filed on Sep. 16, 2005.

(51) Int. Cl. A61F 2/10

(52) U.S. Cl. 623/15.11

(57) ABSTRACT

A method for implanting and retaining hair extensions into human skin, so that they can replace lost natural hair. A hair extension is affixed to a termination on one end. An anchor is then placed over this termination. A channel is cut through the skin's outer layer. An insert having two ends is provided, with attachment features for attaching the insert to an anchor on each of its two ends. An anchor, with the attached termination and hair extension, is preferably attached to one end of the insert. The opposite end of the insert is then slipped into the channel, leaving a small gap around the anchor and the insert so that they can move relative to the channel. Once the opposite end of the insert arrives at the far end of the channel, a second anchor, with its attached termination and hair extension, is attached to the opposite end. The insert remains in the channel, with the two anchors affixed to its two ends.

### BACKGROUND OF THE INVENTION

1. Field of the Invention

This invention relates to the field of hair replacement. More specifically, the invention comprises a method for implanting hair extensions into the human scalp in order to create a permanent or semi-permanent replacement for lost natural hair.

2. Description of the Related Art

FIG. 1 shows a simplified depiction of a natural hair 18. The hair extends through epidermis 14 down to root 16. Epidermis 14, the outer layer of the skin, can be said to divide the space around the hair into body exterior 10 and body interior 12.

While a discussion of the anatomy and function of the human skin is well beyond the scope of this disclosure, one can generally state that the skin protects the inner organs, bones, muscles, and blood vessels. It regulates the temperature, provides sensory information, and eliminates body wastes through its many pores.

One of the primary functions of the skin is sealing the body in order to create the aforementioned body interior and body exterior. When the skin is pierced, cell growth is stimulated to restore the separation between body interior and body exterior. The piercing of a human earlobe for decorative purposes is a good example. If the earlobe is pierced and nothing is placed in the hole, the hole tends to seal itself and skin will grow back over the entrance and exit wounds (including some scar tissue).

If, on the other hand, a foreign object such as an earring post is placed in the hole, skin will tend to grow around the hole's interior surface. If the foreign object remains in place, the skin will completely cover the inside of the hole and join to the skin on the front and back surfaces of the earlobe.

These two outcomes can be viewed as variations of the skin's natural tendency to encase the body within an unbroken enclosure. In either outcome, the volume surrounding the “intrusion” to the skin's natural state is redivided into a body interior and a body exterior. The present invention seeks to take advantage of this natural phenomenon.

### BRIEF SUMMARY OF THE PRESENT INVENTION

The present invention comprises a method for implanting and retaining hair extensions into human skin, so that they can replace lost natural hair. A hair extension is affixed to a termination on one end. An anchor is then placed over this termination. A channel is cut through the skin's outer layer. An insert having two ends is provided, with attachment features for attaching the insert to an anchor on each of its two ends. An anchor, with the attached termination and hair extension, is preferably attached to one end of the insert. The opposite end of the insert is then slipped into the channel, leaving a small gap around the anchor and the insert so that they can move relative to the channel.

Once the opposite end of the insert arrives at the far end of the channel, a second anchor, with its attached termination and hair extension, is attached to the opposite end. The insert remains in the channel, with the two anchors affixed to its two ends.

As the patient combs his or her hair, the motion of the hair extension moves the anchors and the insert within the channel. This action cleans foreign matter from within the channel and promotes the growth of skin around the walls of the channel. The skin grows until it completely covers the walls of the channel. Once the skin growth is complete, all the implanted components are on the skin's exterior. In other words, though mechanically captured, they are all located in the body exterior. Since there is no intrusion through the skin, the risk of complications inherent in the prior art is greatly reduced.

### BRIEF DESCRIPTION OF THE SEVERAL VIEWS OF THE DRAWINGS

FIG. 1 is an elevation view, showing a natural human hair.

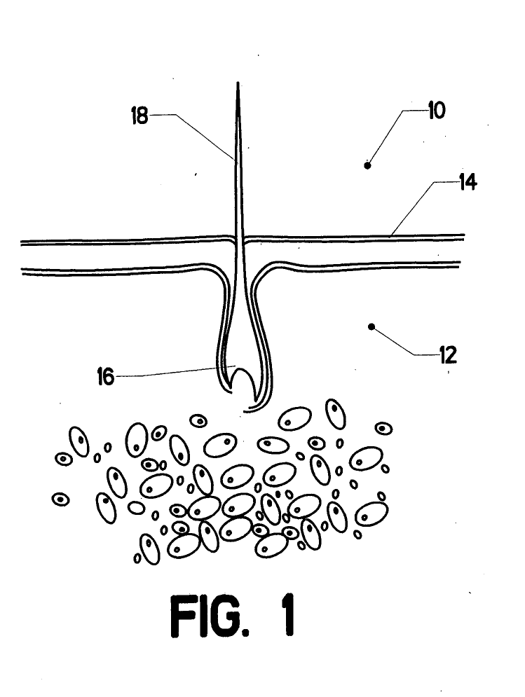

FIG. 2 is a perspective view, showing a variety of hair extensions and terminations.

FIG. 3 is a sectioned perspective view, showing the attachment of an anchor to a hair extension.

FIG. 3B is a perspective view, showing an insert.

FIG. 4 is an elevation view, showing the preparation of a channel into the skin.

FIG. 4B is an elevation view, showing the insertion of the insert into the channel.

FIG. 5 is a sectioned elevation view, showing the hair extension in place.

FIG. 6 is a sectioned elevation view, showing the growth of skin around the hair extension.

### REFERENCE NUMERALS IN THE DRAWINGS

10 body exterior
12 body interior
14 epidermis
16 root
18 natural hair
20 channel
22 hair extension
24 conical termination
26 cylindrical termination
28 spherical termination
30 anchor
32 gap
34 recess
36 insert
38 snap feature

### DESCRIPTION OF THE INVENTION

Those skilled in the art will know that hair extensions have long been available for augmentation of thinning hair. Such hair extensions are typically made of human hair, animal hair, or synthetic fibers. These are typically interwoven with the remaining natural hair to create a temporary hair loss solution. Of course, the interweaving must be regularly renewed.

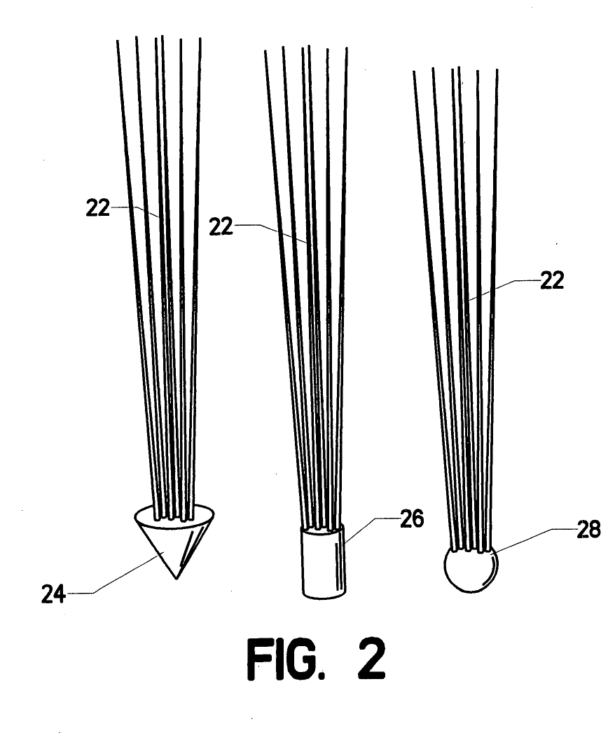

FIG. 2 shows three clusters of hair extension similar to those used for prior art hair augmentation. These have been modified, however, by locking a cluster of hair extensions 22 into a termination on one end. The termination is affixed to one end of each cluster of hair by any suitable bonding method (such as gluing, molding the termination around the hair, etc.). The terminations can assume a variety of forms. Conical termination 24, cylindrical termination 26, and spherical termination 28 are shown as examples. A variety of other shapes could be used as well. The important concept is that the termination is affixed to the cluster of hair and provides a suitable engagement feature for attaching the hair to something else.

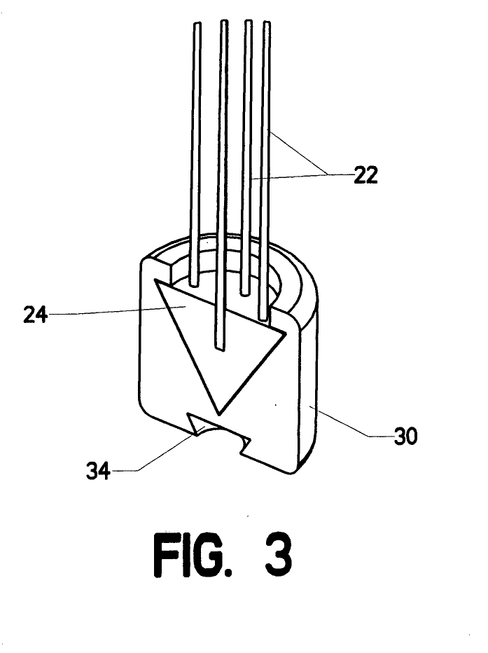

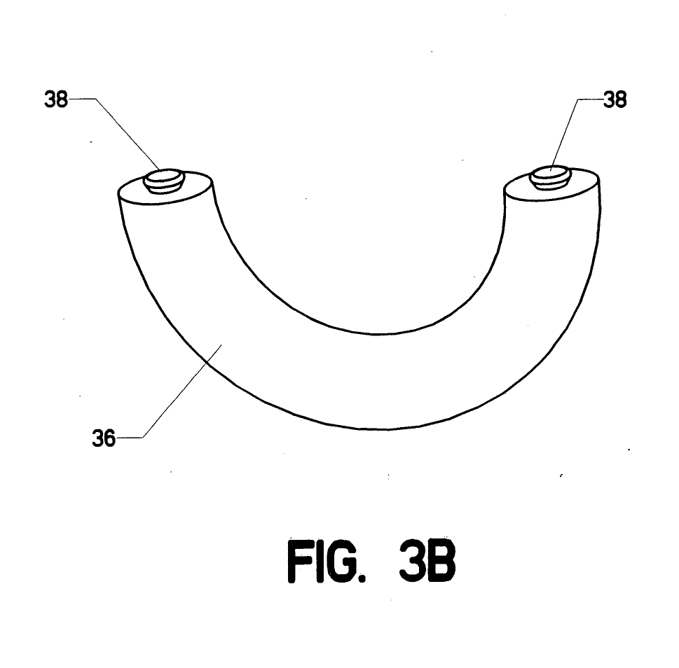

FIG. 3 is a section view, showing the attachment of cleaning anchor 30 over the end of conical termination 24. Anchor 30 can be made of any suitable material. Examples include stainless steel, various high performance plastics, and other natural and synthetic products. The cleaning anchor can be attached to the termination by any suitable method, including gluing, mechanical swaging, melt-bonding, etc. The result is an assembly of hair extension 22, conical termination 24, and anchor 30. The reader will note that anchor 30 covers the end portion of the hair. The reason for this will be made apparent. The reader will also note that the anchor's base includes a suitable mechanical attachment feature. The embodiment shown includes recess 34, which is a radial dovetail.

FIG. 3B shows insert 36, which can be made in the half-ring shape shown. It has two ends, each of which includes a snap feature 38. This snap feature is designed to mechanically interlock with the recess 34 on the bottom of each anchor 30. If an anchor is pressed onto an exposed end of insert 36, the anchor will snap onto the snap feature and lock the two components together. Thus, a hair extension assembly can be locked to either end of insert 36.

Of course, the snap feature shown is one option among many possible mechanical interlocking features. The components could be joined by threads, adhesives, melting processes, etc. The choice of interlocking feature may well dictate the material selection for the insert. The insert can be stainless steel, NYLON, or other moldable plastics. The same can be said for the anchor.

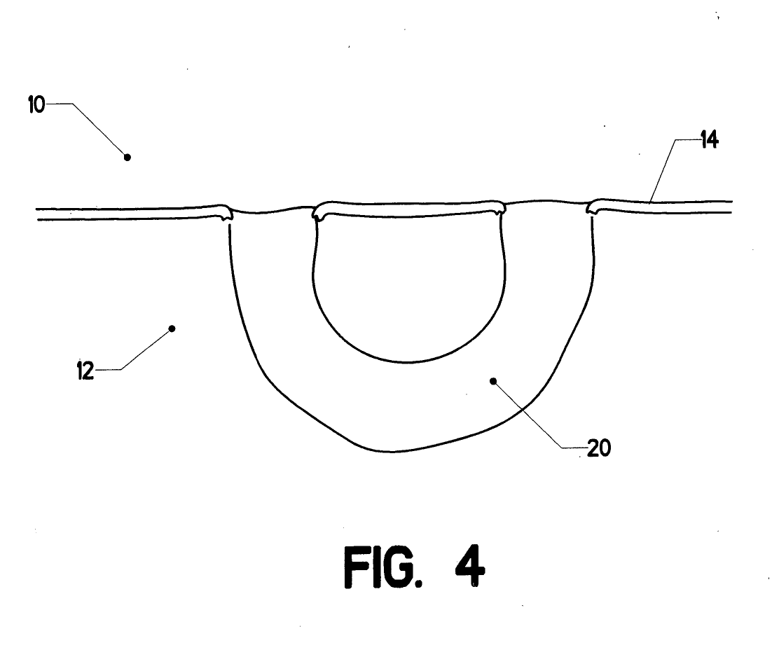

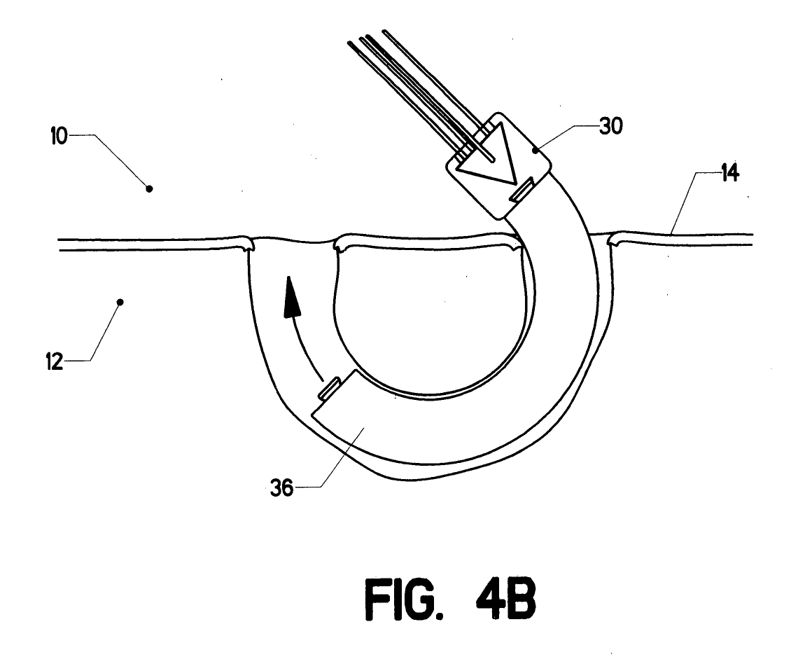

The insert is intended to be inserted into the patient's skin. First, however, the skin must be suitably prepared. A channel must be cut into the epidermis. FIG. 4 is an elevation view of such a channel. An instrument of suitable design has been used to pierce epidermis 14 and create channel 20. Two openings have thereby been created between body exterior 10 and body interior 12.

FIG. 4B shows the placement of the insert within the channel. An anchor is preferably attached to the trailing end of the insert prior to placing the assembly in the channel. Insert 36 is then slipped into the channel until the leading end lies proximate the second opening to the channel. A second anchor—with attached hair extension—is then affixed to the leading end of the insert.

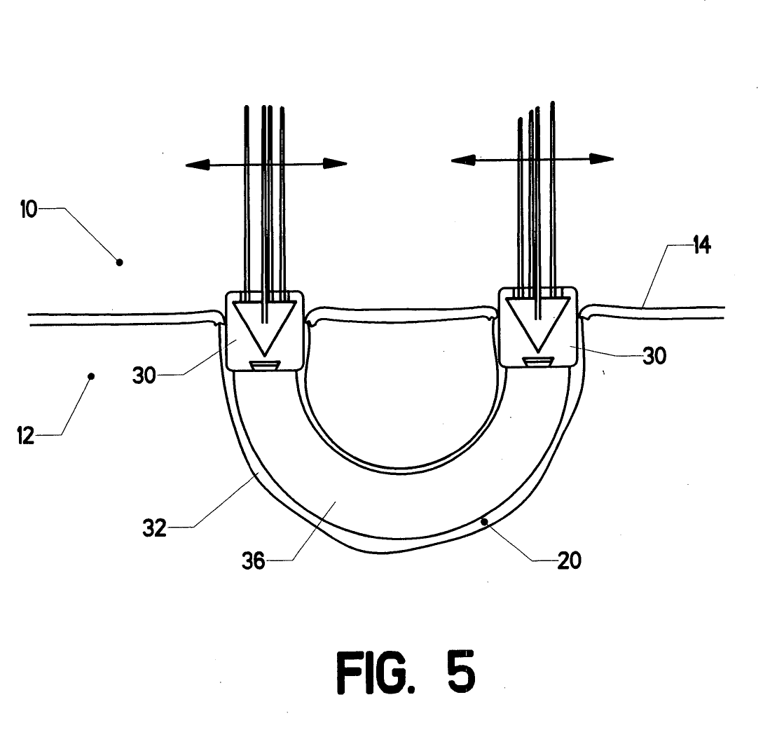

The result is shown in FIG. 5. Insert 36 lies within channel 20. The two anchors are preferably enlarged with respect to the insert so that they will not tend to slip back into the channel. In other words, the combination of the insert and the two anchors creates a stable placement of the assembly within the channel.

The channel is sized so that gap 32 remains around the insert (The gap is small, typically less than 0.5 mm). If the hair is moved back and forth in the directions indicated by the arrow—typically by brushing the hair in a natural fashion—the gap allows the insert to move about within the channel. This action prevents the adhesion of unwanted substances within the channel which might cause infection. It may also stimulate the growth of the skin around the channel.

Returning briefly to FIG. 4B, the reader will observe that it is possible to permanently affix a hair replacement assembly to one end of the insert while still allowing the insert to be installed as shown. Thus, one need not include mechanical interlocking features on both ends of the insert. One could permanently attach a hair assembly to one end of the insert (even molding them as one unit). This is simply a design choice.

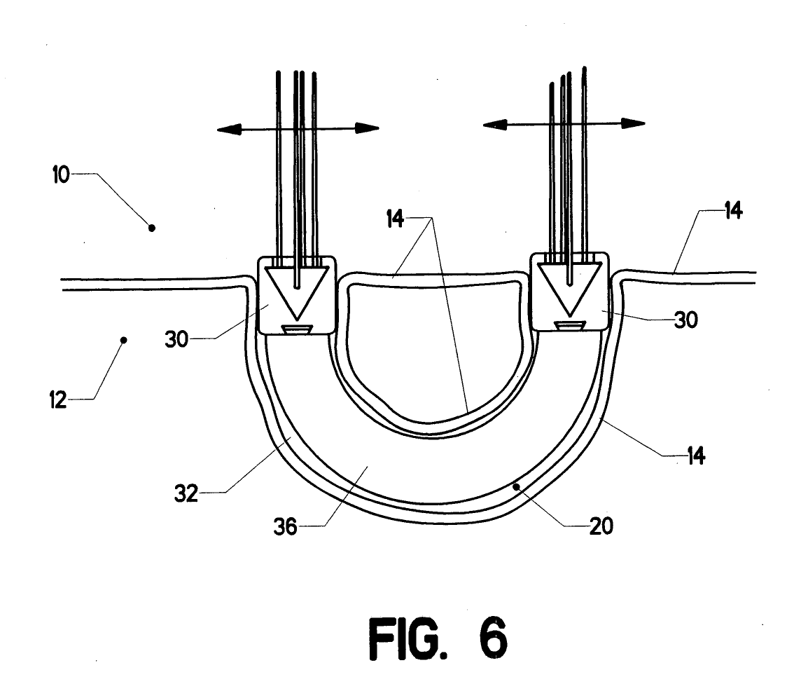

Over time the skin will tend to grow around the insert and the anchors. FIG. 6 shows this process after it is complete. Epidermis 14 now completely surrounds the implanted assembly (lining the tubular wall of the channel, as for a pierced earlobe). Significantly, the skin has displayed its normal reaction to intrusion. It has redivided the volume into the body exterior and the body interior. The reader will note that all the implanted components lie on the body exterior (outside the skin). Although they are mechanically captured, they lie outside the epidermis. Thus, there is little risk of infection.

The reader will therefore understand that the present inventive process allows the implantation and mechanical capture of replacement hair without the need to permanently pierce the body's skin. The process uses only a transitory piercing. It then takes advantage of the skin's natural closing tendency to mechanically capture the components in a permanent or semi-permanent installation.

These descriptions contain significant detail regarding the novel aspects of the present invention. They should not be construed, however, as limiting the scope of the invention but rather as providing illustrations of the preferred embodiments of the invention. Thus, the scope of the invention should be fixed by the claims, rather than by the examples given.

1. A hair replacement system for affixing hair to the human skin using a curved channel cut through said skin, wherein said channel has at least a first opening, comprising:
a. a first anchor, sized to fit within said first opening;
b. a curved insert attached to said first anchor, wherein said curved insert is sized to fit loosely within said curved channel so that said curved insert is able to move about within said channel; and
c. at least one hair extension attached to said first anchor.

2. A hair replacement system as recited in claim 1, wherein said first anchor is large enough to prevent its passage into said curved channel.

3. A hair replacement system as recited in claim 1, wherein said first anchor is detachable from said curved insert.

4. A hair replacement system as recited in claim 1, wherein said anchor comprises:
a. a first material which is attached to said at least one hair extension; and
b. a second material which surrounds a substantial portion of said first material.

5. A hair replacement system as recited in claim 1, wherein said curved channel includes a second opening, and wherein said hair replacement system further comprises:
a. a second anchor, sized to fit within said second opening; and
b. a detachable attachment between said second anchor and said curved insert.

6. A hair replacement system as recited in claim 5, wherein said second anchor is large enough to prevent its passage into said curved channel.

7. A hair replacement system as recited in claim 5, wherein said second anchor comprises:
a. a first material which is attached to said at least one hair extension; and
b. a second material which surrounds a substantial portion of said first material.

8. A hair replacement system as recited in claim 4, wherein said second material is attached to said first material by a method selected from the group consisting of gluing, mechanical swaging, or melt-bonding.

9. A hair replacement system as recited in claim 8, wherein said second material is attached to said first material by a method selected from the group consisting of gluing, mechanical swaging, or melt-bonding.

10. A hair replacement system as recited in claim 1, wherein said at least one hair extension is attached to said first anchor by casting a portion of said first anchor over a portion of said at least one hair extension.

11. A hair replacement system as recited in claim 2, wherein said at least one hair extension is attached to said first anchor by casting a portion of said first anchor over a portion of said at least one hair extension.

12. A hair replacement system as recited in claim 3, wherein said at least one hair extension is attached to said first anchor by casting a portion of said first anchor over a portion of said at least one hair extension.

13. A hair replacement system as recited in claim 4, wherein said at least one hair extension is attached to said first anchor by casting a portion of said first anchor over a portion of said at least one hair extension.

14. A hair replacement system as recited in claim 2, wherein said anchor comprises:
a. a first material which is attached to said at least one hair extension; and
b. a second material which surrounds a substantial portion of said first material.

15. A hair replacement system as recited in claim 3, wherein said anchor comprises:
a. a first material which is attached to said at least one hair extension; and
b. a second material which surrounds a substantial portion of said first material.

16. A hair replacement system as recited in claim 6, wherein said second anchor comprises:
a. a first material which is attached to said at least one hair extension; and
b. a second material which surrounds a substantial portion of said first material.

17. A hair replacement system as recited in claim 1, wherein said first anchor is detachably attached to said curved insert by a mechanically interlocking snap feature.

18. A hair replacement system as recited in claim 5, wherein said second anchor is detachably attached to said curved insert by a mechanically interlocking snap feature.

19. A hair replacement system as recited in claim 2, wherein said first anchor is detachably attached to said curved insert by a mechanically interlocking snap feature.

20. A hair replacement system as recited in claim 6, wherein said second anchor is detachably attached to said curved insert by a mechanically interlocking snap feature.
